(19) United States
(12) Patent Application Publication
(10) Pub. No.: US 2026/0306476 A1
(43) Pub. Date: Oct. 8, 2026

(54) ANTIBACTERIAL COMPOUND PBT2

(71) Applicant: GRIFFITH UNIVERSITY, Nathan, Queensland (AU)

(72) Inventors: Michael JENNINGS, Nathan, Queensland (AU); Mark VON ITZSTEIN, Nathan, Queensland (AU); Ibrahim Mustafa EL-DEEB, Nathan, Queensland (AU); Freda JEN, Nathan, Queensland (AU)

(21) Appl. No.: 19/100,799

(22) PCT Filed: Jan. 18, 2023
(86) PCT No.: PCT/AU2023/050023

§ 371 Date: Feb. 3, 2025

(30) Foreign Application Priority Data
Aug. 1, 2022 (AU) 2022902157

(51) Int. Cl. A61K 31/47; A61P 31/04

(52) U.S. Cl. CPC A61K 31/47; A61P 31/04

(57) ABSTRACT

The present disclosure relates to methods, uses and pharmaceutical compositions for the treatment and/or prophylaxis of bacterial infections caused by a bacteria selected from the group consisting of Neisseria gonorrhoeae, Neisseria meningitidis, nontypeable Haemophilus influenzae, Campylobacter and Helicobacter pylori, comprising administering an effective amount of PBT2.

### TECHNICAL FIELD

This disclosure relates to methods, uses and pharmaceutical compositions for the treatment and/or prophylaxis of bacterial infections caused by a bacteria selected from the group consisting of Neisseria gonorrhoeae, Neisseria meningitidis, nontypeable Haemophilus influenzae, Campylobacter and Helicobacter pylori.

### BACKGROUND

The emergence and spread of multidrug resistant (MDR) pathogens is a pressing global health concern. Antimicrobial resistance (AMR) happens when pathogens, such as bacteria, viruses, fungi and parasites, develop a resistance to medicines that have been traditionally used to kill them. This makes it increasingly difficult to treat infections and it increases the risk of spread of the pathogen, severe illness and death. Because of this drug resistance, first-line antimicrobial drugs lose their efficacy against the pathogens.

Antibacterial resistance occurs when bacteria develop the ability to resist antibiotics. These multi- and pan-resistant bacteria, also known as ‘superbugs’, cause infections that cannot be appropriately treated with existing antibiotics. There is therefore a pressing need to develop new methodologies to treat bacterial infections caused by multidrug resistant bacteria.

### SUMMARY

In a first aspect, the present disclosure relates to a method for the treatment and/or prophylaxis a bacterial infection selected from the group consisting of: Neisseria gonorrhoeae, Neisseria meningitidis, nontypeable Haemophilus influenzae, Campylobacter and Helicobacter pylori infections, in a subject, comprising administering an effective amount of PBT2, or a pharmaceutically acceptable salt thereof, to the subject.

In a second aspect, the present disclosure relates to the use of PBT2, or a pharmaceutically acceptable salt thereof, in the preparation of a medicament for the treatment and/or prophylaxis of a bacterial infection selected from the group consisting of: Neisseri agonorrhoeae, Neisseria meningitidis, nontypeable Haemophilus influenzae, Campylobacter and Helicobacter pylori infections.

In a third aspect, the present disclosure relates to the use of PBT2, or a pharmaceutically acceptable salt thereof, for the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections.

In a fourth aspect, the present disclosure relates to PBT2, or a pharmaceutically acceptable salt thereof, for use in the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections, in a subject.

In a fifth aspect, the present disclosure relates to a pharmaceutical composition comprising PBT2, or a pharmaceutically acceptable salt thereof, for use in the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections, in a subject.

In a sixth aspect, the present disclosure relates to a method for the treatment and/or prophylaxis of a bacterial infection selected from the group consisting of: Neisseria meningitidis, nontypeable Haemophilus influenzae, Campylobacter and Helicobacter pylori infections, in a subject, comprising administering an effective amount of PBT2, or a pharmaceutically acceptable salt thereof, to the subject.

In a seventh aspect, the present disclosure relates to the use of PBT2, or a pharmaceutically acceptable salt thereof, in the preparation of a medicament for the treatment and/or prophylaxis of a bacterial infection selected from the group consisting of: Neisseria meningitidis, nontypeable Haemophilus influenzae, Campylobacter and Helicobacter pylori infections.

In an eighth aspect, the present disclosure relates to the use of PBT2, or a pharmaceutically acceptable salt thereof, for the treatment and/or prophylaxis of a bacterial infection selected from the group consisting of: Neisseria meningitidis, nontypeable Haemophilus influenzae, Campylobacter and Helicobacter pylori infections.

In a ninth aspect, the present disclosure relates to PBT2, or a pharmaceutically acceptable salt thereof, for use in the treatment and/or prophylaxis of a bacterial infection selected from the group consisting of: Neisseria meningitidis, nontypeable Haemophilus influenzae, Campylobacter and Helicobacter pylori infections, in a subject.

In a tenth aspect, the present disclosure relates to a pharmaceutical composition comprising PBT2, or a pharmaceutically acceptable salt thereof, for use in the treatment and/or prophylaxis of a bacterial infection selected from the group consisting of: Neisseria meningitidis, nontypeable Haemophilus influenzae, Campylobacter and Helicobacter pylori infections, in a subject.

In some embodiments of the first to tenth aspects, PBT2, or a pharmaceutically acceptable salt thereof, is the sole active antibacterial agent.

In some embodiments of the first to tenth aspects, PBT2, or a pharmaceutically acceptable salt thereof, is administered, or is formulated for administration, in the absence of zinc.

In some embodiments of the first to tenth aspects, PBT2, or a pharmaceutically acceptable salt thereof, is administered, or is formulated for administration, in the absence of a further antibacterial agent.

In a preferred embodiment, PBT2, or a pharmaceutically acceptable salt thereof, is administered, or is formulated for administration, in the absence of zinc and a further antibacterial agent.

In some embodiments, a dose of PBT2, or a pharmaceutically acceptable salt thereof, administered to the subject, or formulated for administration to the subject, is between about 1 mg and about 250 mg per day, between about 1 mg and about 200 mg per day, between about 1 mg and about 150 mg per day, between about 1 mg and about 100 mg per day, between about 1 mg and about 50 mg per day, or between about 1 mg and about 30 mg per day.

In some embodiments, PBT2, or a pharmaceutically acceptable salt thereof, is administered to the subject, or is formulated for administration to the subject, in an amount between about 1 mg and about 250 mg per day, between about 1 mg and about 200 mg per day, between about 1 mg and about 150 mg per day, between about 1 mg and about 100 mg per day, between about 1 mg and about 50 mg per day, or between about 1 mg and about 30 mg per day.

In an eleventh aspect, the present disclosure relates to a pharmaceutical composition comprising an effective amount of PBT2, or a pharmaceutically acceptable salt thereof, and a pharmaceutically acceptable excipient wherein the composition does not comprise zinc or a further antibacterial active.

In some embodiments of the eleventh aspect, the pharmaceutical composition consists of or consists essentially of PBT2, or a pharmaceutically acceptable salt thereof, and one or more pharmaceutically acceptable excipients, diluents or carriers.

### BRIEF DESCRIPTION OF DRAWINGS

FIG. 1: Time-kill curves of Neisseria gonorrhoeae WHO Z in GC broth with or without PBT2. Error bars indicate standard deviation from 3 biological replicates.

FIG. 2: Antibiotic resistance development assay of PBT2. Kanamycin (control) was used to show the development of resistance to kanamycin over 30 days of treatment.

FIG. 3: Volcano plot of differentially expressed genes between bacteria treated with/without PBT2.

FIG. 4: Chart indicating PBT2 can cure Neisseria gonorrhoeae infection of human cervical (Pex) cells. PBT2 treatment resulted in a dose-dependent reduction in Neisseria gonorrhoeae strain WHO Z survival at 24 h post-treatment. Ceftriaxone (CRO, positive control) is currently recommended for gonorrhoea treatment. Veh—0.1% DMSO vehicle control; UT—untreated; * p<0.0001 versus vehicle for CRO or PBT2 treatment.

### DESCRIPTION OF EMBODIMENTS

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the present disclosure includes all such variations and modifications. The present disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the present disclosure.

Any embodiment of the present disclosure herein shall be taken to apply mutatis mutandis to any other embodiment of the present disclosure unless specifically stated otherwise.

Unless specifically defined otherwise, all technical and scientific terms used herein shall be taken to have the same meaning as commonly understood by one of ordinary skill in the art (for embodiments, in microbiology including bacteriology, organic synthetic chemistry, cell culture, molecular genetics, medicinal chemistry, and biochemistry).

The term “and/or”, e.g., “X and/or Y” shall be understood to mean either “X and Y” or “X or Y” and shall be taken to provide explicit support for both meanings or for either meaning.

The terms “from” and “to”, when indicating a range, shall be understood to mean the range is inclusive of the recited lower and upper values. For example, “x is an integer from 0 to 6” shall be understood as including the situation in which x is not present (x is 0), that in which x is 6, as well as each whole number integer value in between, i.e. wherein x is 1, 2, 3, 4, or 5.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the present disclosure for the listed elements. Thus, the phrase “consisting essentially of” indicates that the listed elements are required or mandatory, but that other elements are optional and may or may not be present depending upon whether or not they affect the activity or action of the listed elements.

### Selected Definitions

As used herein, “effective amount” refers to the administration of an amount of the relevant active agent, PBT2, or a pharmaceutically acceptable salt thereof, sufficient to prevent the occurrence of symptoms of the condition being treated, or to bring about a halt in the worsening of symptoms or to treat and alleviate or at least reduce the severity of the symptoms. In particular to treat and resolve the recited bacterial infection. The effective amount will vary in a manner which would be understood by a person of skill in the art with patient age, sex, weight etc. An appropriate dosage or dosage regime can be ascertained through routine trial.

“Pharmaceutically acceptable carrier, diluent or excipient”, or like terms, refers to any ingredient other than the compounds described herein (for example, a vehicle capable of suspending, complexing, or dissolving the active compound) and having the properties of being substantially nontoxic in a subject. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxy toluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

“Pharmaceutically acceptable salt” includes both acid and base addition salts of PBT2. Lists of suitable salts may be found in Remington's Pharmaceutical Sciences, 18th ed., Mack Publishing Company, Easton, PA, 1990, p. 1445, and Journal of Pharmaceutical Science, 66, 2-19 (1977). Acid addition salts are those which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like. Base addition salts are those which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like.

As used herein, the terms “subject” or “individual” or “patient” may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human in need of treatment and/or prophylaxis for a bacterial infection selected from the group consisting of: Neisseria gonorrhoeae, Neisseria meningitidis, nontypeable Haemophilus influenzae, Campylobacter and Helicobacter pylori infections or a human in need of treatment and/or prophylaxis for a bacterial infection selected from the group consisting of: Neisseria meningitidis, nontypeable Haemophilus influenzae, Campylobacter and Helicobacter pylori infections. However, it will be understood that the aforementioned terms do not imply that symptoms are necessarily present.

As used herein, the terms “treatment” or “treating” of a subject include the application or administration of PBT2, or a pharmaceutically acceptable salt thereof, as described herein to a subject with the purpose of delaying, slowing, stabilizing, curing, healing, alleviating, relieving, altering, remedying, less worsening, ameliorating, improving, or affecting the bacterial infection, the symptom(s) of the bacterial infection, or the risk of (or susceptibility to) the bacterial infection. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, the terms “prophylaxis”, “prevention” or “preventing” are intended to refer to at least the reduction of likelihood of the risk of (or susceptibility to) acquiring a bacterial infection (i.e., causing at least one of the clinical symptoms of the bacterial infection not to develop in a patient that may be exposed to or predisposed to the bacterial infection but does not yet experience or display symptoms of the bacterial infection). Biological and physiological parameters for identifying such patients are provided herein and are known in the art.

It will be appreciated by the person of skill in the art that PBT2 is the trivial name for 5,7-dichloro-2-[(dimethylamino)methyl]quinolin-8-ol and both are accepted terms for the same active pharmaceutical ingredient. References herein to PBT2 can be considered references to 5,7-dichloro-2-[(dimethylamino)methyl]quinolin-8-ol. That is, the terms may be used interchangeably herein.

PBT2 is a 8-hydroxyquinoline derivative and can be synthesised following literature-known procedures (see, for example, Bohlmann L, De Oliveira D M P, El-Deeb I M, Brazel E B, Harbison-Price N, Ong C-L Y, Rivera-Hernandez T, Ferguson S A, Cork A J, Phan M-D, Soderholm A T, Davies M R, Nimmo G R, Dougan G, Schembri M A, Cook G M, McEwan A G, von Itzstein M, McDevitt C A, Walker M J. 2018. Chemical synergy between ionophore PBT2 and zinc reverses antibiotic resistance. mBio 9:e02391-18. https://doi.org/10.1128/mBio.02391-18, which is hereby incorporated by reference in its entirety).

PBT2 has the following structure and may, of course, be obtained in various salt forms if required:

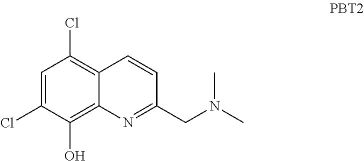 [SMILES: CN(C)Cc1ccc2c(Cl)cc(Cl)c(O)c2n1]

PBT2 acts as an iron carrier, also known as an ionophore. Ionophores can reversibly bind to ions and transport them across biological membranes. Because of these properties, they have been of interest in the development of new drugs.

The ionophore PBT2 can carry cations, specifically Zn²⁺, across the blood-brain barrier where it modulates metal ions concentrations. It was originally developed as a potential therapeutic for Alzheimer's and Huntington's disease and has been tested in phase 2 clinical trials. It has been reported to be safe and well-tolerated, without serious side effects or withdrawal symptoms, when taken daily at a dose of 250 mg for up to 12 months.

Studies have shown that PBT2, in the presence of zinc, has the ability to make a variety of gram-positive and gram-negative bacteria more susceptible to antibiotics. It has been reported that it is essential to use PBT2 in combination with zinc and the known antibiotic to achieve an antibacterial effect. A drawback of this approach is that it is still necessary to treat the patient with an antibiotic which may come with known side effects, such as vomiting, nausea, diarrhoea, bloating, indigestion and abdominal pain, as well as the threat of an allergic reaction or anaphylaxis. Further, known and widely used antibiotics may become less efficacious with extensive use as bacterial resistance develops.

Further research (mBio 9:e02391-18. https://doi.org/10.1128/mBio.02391-18, as above, and Harbison-Price N, et al. 2020. Multiple bactericidal mechanisms of the zinc ionophore PBT2. mSphere 5:e00157-20. https://doi.org/10.1128/mSphere. 00157-20) has shown that a PBT2/zinc combination, in the absence of an antibiotic, shows antibacterial activity against a few selected gram-positive bacterial strains, such as the bovine mastitis-causing pathogen Streptococcus uberis (S. uberis), erythromycin-resistant group A Streptococcus (GAS), methicillin-resistant Staphylococcus aureus (MRSA), and vancomycin-resistant Enterococcus (VRE). It was reported that neither PBT2 nor zinc alone displayed antibacterial activity which would be suitable to treat an infection caused by any of the tested bacterial strains and so only the PBT2/Zinc combination had treatment efficacy.

The present inventors have surprisingly identified that PBT2, or a pharmaceutically acceptable salt thereof, alone, in the absence of any additional antibacterial agent and/or zinc, can be used to successfully treat certain bacterial infections. This is particularly surprising in light of previous findings that even high doses of PBT2 were not sufficient to treat infections caused by S. uberis, GAS, MRSA and VRE, as discussed above. Accordingly, the present disclosure provides methods, uses and compositions to treat bacterial infections selected from the group consisting of: Neisseria gonorrhoeae, Neisseria meningitidis, nontypeable Haemophilus influenzae, Campylobacter (the Campylobacter genus; especially pathogenic Campylobacter species) and Helicobacter pylori infections, with PBT2. The inventors have found that PBT2 alone, in the absence of an additional antibacterial agent/antibiotic can be sufficient to successfully treat these particular infections. Further, the inventors have found that a surprisingly low concentration of PBT2 is sufficient for this treatment as shown in the Examples. This could not have been expected from earlier publications, which show little effect on different bacterial species following treatment with high concentrations of PBT2.

Without wishing to be bound by theory, the inventors hypothesise that Neisseria gonorrhoeae, Neisseria meningitidis, nontypeable Haemophilus influenzae, Campylobacter and Helicobacter pylori infections may be particularly susceptible to PBT2 sole antibacterial treatment whereas other bacteria may not demonstrate this susceptibility.

In a first aspect, the present disclosure relates to a method for the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections, in a subject, comprising administering an effective amount of PBT2, or a pharmaceutically acceptable salt thereof, to the subject.

In a second aspect, the present disclosure relates to the use of PBT2, or a pharmaceutically acceptable salt thereof, in the preparation of a medicament for the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections.

In a third aspect, the present disclosure relates to the use of PBT2, or a pharmaceutically acceptable salt thereof, for the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections.

In a fourth aspect, the present disclosure relates to PBT2, or a pharmaceutically acceptable salt thereof, for use in the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections, in a subject.

In a fifth aspect, the present disclosure relates to a pharmaceutical composition comprising PBT2, or a pharmaceutically acceptable salt thereof, for use in the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections, in a subject.

In some embodiments of the first to fifth aspects, the bacterial infection is selected from the group consisting of: Neisseria meningitidis, nontypeable Haemophilus influenzae, Campylobacter and Helicobacter pylori infections.

In a sixth aspect, the present disclosure relates to a method for the treatment and/or prophylaxis of a bacterial infection selected from the group consisting of: Neisseria meningitidis, nontypeable Haemophilus influenzae, Campylobacter and Helicobacter pylori infections, in a subject, comprising administering an effective amount of PBT2, or a pharmaceutically acceptable salt thereof, to the subject.

In a seventh aspect, the present disclosure relates to the use of PBT2, or a pharmaceutically acceptable salt thereof, in the preparation of a medicament for the treatment and/or prophylaxis of a bacterial infection selected from the group consisting of: Neisseria meningitidis, nontypeable Haemophilus influenzae, Campylobacter and Helicobacter pylori infections.

In an eighth aspect, the present disclosure relates to the use of PBT2, or a pharmaceutically acceptable salt thereof, for the treatment and/or prophylaxis of a bacterial infection selected from the group consisting of: Neisseria meningitidis, nontypeable Haemophilus influenzae, Campylobacter and Helicobacter pylori infections.

In a ninth aspect, the present disclosure relates to PBT2, or a pharmaceutically acceptable salt thereof, for use in the treatment and/or prophylaxis of a bacterial infection selected from the group consisting of: Neisseria meningitidis, nontypeable Haemophilus influenzae, Campylobacter and Helicobacter pylori infections, in a subject.

In a tenth aspect, the present disclosure relates to a pharmaceutical composition comprising PBT2, or a pharmaceutically acceptable salt thereof, for use in the treatment and/or prophylaxis of a bacterial infection selected from the group consisting of: Neisseria meningitidis, nontypeable Haemophilus influenzae, Campylobacter and Helicobacter pylori infections, in a subject.

The Neisseria gonorrhoeae, Neisseria meningitidis, nontypeable Haemophilus influenzae, Campylobacter and Helicobacter pylori bacterial infections may be any suitable species (in the case of the Campylobacter genus) and strain(s) of these bacteria known in the art, including those described herein. Examples of suitable Neisseria gonorrhoeae strains include WHO C, WHO E, WHO F, WHO G, WHO K, WHO L, WHO M, WHO N, WHO O, WHO P, WHO X, WHO Y, WHO Z, MS11 and ATCC49226. Examples of suitable Neisseria meningitides strains include Z2491 (A), MC58 (B), 8013 (C), PMC1 (X), PMC2 (Z/29E), PMC10 (Y) and PMC19 (W). Examples of suitable non-typeable Haemophilus influenzae strains include 2019, 723, 86-028NP, R2866 and ATCC49766. Examples of suitable Campylobacter species include pathogenic Campylobacter species, for example Campylobacter jejuni, Campylobacter coli, Campylobacter abortis, Campylobacter hepaticas, Campylobacter upsaliensis, Campylobacter helveticus and Campylobacter fetus (especially Campylobacter fetus subspecies fetus). Examples of suitable Campylobacter jejuni strains include 81-176, NCTC 11168 and ATCC 33560. Examples of suitable Helicobacter pylori strains include 26695, ATCC 43504, CH426 and CH428.

In some embodiments of the present disclosure, the Campylobacter is selected from the group consisting of Campylobacter jejuni, Campylobacter coli, Campylobacter abortis, Campylobacter hepaticas, Campylobacter upsaliensis, Campylobacter helveticus and Campylobacter fetus (especially Campylobacter fetus subspecies fetus). In some embodiments, the Campylobacter is Campylobacter jejuni. In some embodiments, the Campylobacter is selected from the group consisting of Campylobacter coli, Campylobacter abortis, Campylobacter hepaticas, Campylobacter upsaliensis, Campylobacter helveticus and Campylobacter fetus (especially Campylobacter fetus subspecies fetus).

It is a surprising advantage of the present disclosure that PBT2 shows the desired antibacterial properties without the addition of a further known antibiotic agent. Therefore, in some embodiments of the first to tenth aspects, PBT2, or a pharmaceutically acceptable salt thereof, is the sole active antibacterial agent in the treatment.

It is known that PBT2 in combination with zinc has the ability to reverse antibiotic resistance in several bacteria and, in a small number of species, may be bactericidal. However, the inventors have surprisingly discovered that PBT2, or a pharmaceutically acceptable salt thereof, in the absence of zinc can effectively kill certain pathogens. As discussed herein, the inventors hypothesise that these bacterial species, as identified in the present disclosure, are particularly susceptible to PBT2 antibacterial treatment even though other species have proven resistant to sole treatment with PBT2.

In some embodiments of the first to tenth aspects, PBT2, or a pharmaceutically acceptable salt thereof, is administered, or is formulated for administration, in the absence of zinc.

In a preferred embodiment, PBT2, or a pharmaceutically acceptable salt thereof, is administered, or is formulated for administration, in absence of zinc and a further antibacterial agent.

Furthermore, as discussed herein, the inventors have surprisingly found that a much lower dose of PBT2, or a pharmaceutically acceptable salt thereof, can achieve the desired antibacterial effects than would be expected from known combination treatments, as demonstrated in the Examples.

The PBT2, or pharmaceutically acceptable salt thereof, as described herein, may be administered to the subject, or formulated for administration to the subject, in an amount suitable to achieve a blood concentration of at least about 0.005 mg/L up to about 25 mg/L, and all combinations and sub-combinations of ranges therein. For example, the PBT2, or pharmaceutically acceptable salt thereof, may be administered to the subject, or formulated for administration to the subject, in an amount to achieve a blood concentration of at least about 0.005 mg/L, for example at least about 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9 or 1.0 mg/L, up to about 25 mg/L, for example up to about 24, 23, 22, 21, 20, 19, 18, 17, 16, 15, 14, 13, 12, 11, 10, 9, 8, 7, 6, 5, 4 or 3 mg/L. Any minimum and maximum amount may be combined to form a range, provided the range is between 0.005 to 25 mg/L.

In some embodiments of the first to tenth aspects, PBT2, or a pharmaceutically acceptable salt thereof, is administered to the subject, or is formulated for administration to the subject, in an amount to achieve a blood concentration of between about 0.005 to about 25 mg/L, between about 0.005 to about 20 mg/L, between about 0.005 to about 15 mg/L, between about 0.005 to about 10 mg/L, between about 0.005 to about 5 mg/L, between about 0.005 to about 4 mg/L, between about 0.005 to about 3 mg/L, between about 0.01 to about 25 mg/L, between about 0.01 to about 20 mg/L, between about 0.01 to about 15 mg/L, between about 0.01 to about 10 mg/L, between about 0.01 to about 5 mg/L, between about 0.01 to about 4 mg/L, between about 0.01 to about 3 mg/L, between about 0.5 to about 25 mg/L, between about 0.5 to about 20 mg/L, between about 0.5 to about 15 mg/L, between about 0.5 to about 10 mg/L, between about 0.5 to about 5 mg/L, between about 0.5 to about 4 mg/L, between about 0.5 to about 3 mg/L, between about 1.0 to about 25 mg/L, between about 1.0 to about 20 mg/L, between about 1.0 to about 15 mg/L, between about 1.0 to about 10 mg/L, between about 1.0 to about 5 mg/L, between about 1.0 to about 4 mg/L, or between about 1.0 to about 3 mg/L.

In some embodiments, a dose of PBT2, or a pharmaceutically acceptable salt thereof, administered to the subject, or formulated for administration to the subject, is between about 1 mg and about 250 mg per day, between about 1 mg and about 200 mg per day, between about 1 mg and about 150 mg per day, between about 1 mg and about 100 mg per day, between about 1 mg and about 50 mg per day, or between about 1 mg and about 30 mg per day.

In some embodiments, PBT2, or a pharmaceutically acceptable salt thereof, is administered to the subject, or is formulated for administration to the subject, in an amount between about 1 mg and about 250 mg per day, between about 1 mg and about 200 mg per day, between about 1 mg and about 150 mg per day, between about 1 mg and about 100 mg per day, between about 1 mg and about 50 mg per day, or between about 1 mg and about 30 mg per day.

In some embodiments of the first to tenth aspects, PBT2, or a pharmaceutically acceptable salt thereof, when administered to the subject, will demonstrate at least a 10%, 20%, 30%, 40% or 50% reduction in bacterial infection. This reduction in bacterial infection may be within a period of less than 24 hours, 20 hours, 16 hours, 10 hours, 8 hours, 6 hours, 5 hours or 4 hours.

It will be appreciated that suitable treatment and/or prophylactic regimens with PBT2, or a pharmaceutically acceptable salt thereof, may vary depending on the subject being treated. In some embodiments, the treatment or dose is administered, or is formulated for administration, daily for 7, 6, 5, 4, 3, 2, or 1 day(s). In some embodiments, the treatment or dose is administered, or is formulated for administration, daily for at least 1, 2, 3, 4, 5, 6 or 7 day(s). In some embodiments, the treatment or dose is administered, or formulated for administration, daily for up to 4 weeks, 3 weeks, 2 weeks or 1 week. Any minimum and maximum period may be combined to form a range.

The treatment can be repeated as often as needed. It is one advantage of the present disclosure that the recited pathogens do not appear to develop any resistance to PBT2 over the testing period. As described in the Examples, there was no visible development of resistance to PBT2 during a 30 day period.

Therefore, in certain embodiments of the first to tenth aspects, the treatment and/or prophylaxis using PBT2, or a pharmaceutically acceptable salt thereof, may be treatment and/or prophylaxis of a bacterial infection selected from the group consisting of: Neisseria gonorrhoeae, Neisseria meningitidis, nontypeable Haemophilus influenzae, Campylobacter and Helicobacter pylori infections which is resistant to treatment with one or more known antibiotics. That is, the infection is refractory to at least one standard antibiotic. In some embodiments, the bacterial infection is selected from the group consisting of: Neisseria meningitidis, nontypeable Haemophilus influenzae, Campylobacter and Helicobacter pylori infections which is resistant to treatment with one or more known antibiotics.

Typically, the daily dose can be administered, or formulated for administration, in one to four doses per day. For example, the daily dose may be administered, or formulated for administration, in at least one and up to four doses per day, and all combinations of ranges therein. In some embodiments, the daily dose is administered, or formulated for administration, in one, two, three or four doses per day.

It will be appreciated that the amount of PBT2, or a pharmaceutically acceptable salt thereof, that is administered and the dosage regimen for the treatment and/or prophylaxis of a microbial infection with it, and/or pharmaceutical compositions of the present disclosure depends on a variety of factors, including the age, weight, sex and medical condition of the subject, the severity of the disease, the route and frequency of administration, and the particular compound employed, and thus may vary widely.

In some embodiments of the first to tenth aspects, the treatment and/or prophylaxis may be administered, or formulated for administration, orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In an embodiment, the treatment is administered orally or intravenously. In another embodiment, the treatment is formulated for oral or intravenous administration. In a preferred embodiment, the treatment is administered orally. In another embodiment, the treatment is formulated for oral administration.

The PBT2, or pharmaceutically acceptable salt thereof, as described herein, may be administered, or formulated for administration by, any route described herein. As used herein, the term “administered” means administration of a therapeutically effective dose of PBT2, or a pharmaceutically acceptable salt thereof, as described herein, to the subject. As used herein, the term “formulated for administration” means a therapeutically effective dose of PBT2, or a pharmaceutically acceptable salt thereof, as described herein, is formulated in such a way that is suitable for the route of administration. In an embodiment, the PBT2, or pharmaceutically acceptable salt thereof, is administered orally or intravenously. In another embodiment, the PBT2, or pharmaceutically acceptable salt thereof, is formulated for oral or intravenous administration. In a preferred embodiment, the PBT2, or pharmaceutically acceptable salt thereof, is administered orally. In another embodiment, the PBT2, or pharmaceutically acceptable salt thereof, is formulated for oral administration.

In some embodiments of the first to tenth aspects, the subject is a vertebrate animal.

In some embodiments of the first to tenth aspects, the subject is a mammal or an avian subject.

In preferred embodiments of the first to tenth aspects, the subject is a human subject in need of treatment and/or prophylaxis for a bacterial infection, especially a bacterial infection selected from the group consisting of: Neisseria gonorrhoeae, Neisseria meningitidis, nontypeable Haemophilus influenzae, Campylobacter and Helicobacter pylori infections. In these embodiments, the Campylobacter is preferably Campylobacter jejuni.

In some embodiments of the first to tenth aspects, the subject is a domestic or livestock animal subject in need of treatment and/or prophylaxis for a bacterial infection, especially a bacterial infection selected from the group consisting of: Neisseria gonorrhoeae, Neisseria meningitidis, nontypeable Haemophilus influenzae, Campylobacter and Helicobacter pylori infections. In these embodiments, the Campylobacter is preferably selected from the group consisting of Campylobacter coli, Campylobacter abortis, Campylobacter hepaticas, Campylobacter upsaliensis, Campylobacter helveticus and Campylobacter fetus (especially Campylobacter fetus subspecies fetus). In some embodiments, the livestock animal may be selected from the group consisting of chicken, cattle, pigs and horse.

In an eleventh aspect, the present disclosure relates to a pharmaceutical composition comprising an effective amount of PBT2, or a pharmaceutically acceptable salt thereof, and a pharmaceutically acceptable excipient, diluent or carrier, wherein the composition does not comprise zinc or a further antibacterial active.

In some embodiments of the eleventh aspect, the pharmaceutical composition is an antibacterial composition which consists of or consists essentially of PBT2, or a pharmaceutically acceptable salt thereof, as the antibacterial active. In this context, “consists of” and “consists essentially of” is intended to mean the pharmaceutical composition includes only PBT2, or a pharmaceutically acceptable salt thereof, as an antibacterial active and does not include any other antibacterial active.

In some embodiments of the eleventh aspect, the pharmaceutical composition consists of or consists essentially of PBT2, or a pharmaceutically acceptable salt thereof, and one or more pharmaceutically acceptable excipients, diluents or carriers.

The pharmaceutical composition as described herein can be formulated to contain PBT2, or a pharmaceutically acceptable salt thereof, in a concentration of at least about 5 wt % up to about 99 wt %, and all combinations and sub-combinations of ranges therein. In some embodiments, the pharmaceutical composition comprises at least 5 wt %, 10 wt %, 15 wt %, 20 wt %, 25 wt %, 30 wt %, 40 wt %, 50 wt %, 60 wt %, 70 wt %, 80 wt %, 90 wt %, or 99 wt % of PBT2. In some embodiments, the pharmaceutical composition comprises no more than 99 wt %, 95 wt %, 90 wt %, 80 wt %, 75 wt %, 70 wt %, 60 wt %, 50 wt %, 40 wt %, 30 wt %, or 20 wt % of PBT2. Thus, the pharmaceutical composition may comprise from 5 wt % to 99 wt % of PBT2. As recognised by the person skilled in the art, the range may be narrowed based on the upper and lower amounts specified above, such as 10 to 95 wt %, 10 to 80 wt %, 15 to 90 wt %, 20 to 75 wt %, 30 to 70 wt %. The pharmaceutical composition may comprise between 20 wt % and 90 wt % of PBT2. The pharmaceutical composition may comprise between 30 wt % and 80 wt % of PBT2. The pharmaceutical composition may comprise between 40 wt % and 70 wt % of PBT2.

In some embodiments, the pharmaceutical composition may be formulated to be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In an embodiment, the treatment is administered orally or intravenously. In a preferred embodiment, the treatment is administered orally.

Contemplated pharmaceutically acceptable compositions may be orally administered in any orally acceptable dosage form including capsules, tablets, troches, elixirs, suspensions, syrups, wafers, chewing gums, aqueous suspensions or solutions. Oral compositions may contain additional ingredients such as: a binder such as microcrystalline cellulose, gum tragacanth or gelatine; an excipient such as starch or lactose, a disintegrating agent such as alginic acid, corn starch and the like; a lubricant such as magnesium stearate; a glidant such as colloidal silicon dioxide; and a sweetening agent such as sucrose or saccharin or flavouring agent such as peppermint, methyl salicylate, or orange flavouring. When the dosage unit form is a capsule, it may additionally contain a liquid carrier such as a fatty oil. Other dosage unit forms may contain other various materials which modify the physical form of the dosage unit, such as, for example, a coating. Thus, tablets or pills may be coated with sugar, shellac, or other enteric coating agents. A syrup may contain, in addition to the active ingredients, sucrose as a sweetening agent and certain preservatives, dyes and colourings and flavours. Materials used in preparing these various compositions should be pharmaceutically pure and non-toxic in the amounts used.

In some embodiments of any of the first to eleventh aspects, PCT2 may be administered, or formulated for administration, in the absence of zinc or a further antibacterial active to up or down-regulate any of the genes, as appropriate, described in Table 3 of the Examples.

The present disclosure also relates to a method for the treatment and/or prophylaxis of a bacterial infection which is associated with the modulation of any one or more of the genes described in Table 3 of the Examples, in a subject, the method comprising administering an effective amount of PBT2, or a pharmaceutically acceptable salt thereof, to the subject.

The present disclosure also relates to use of PBT2, or a pharmaceutically acceptable salt thereof, in the preparation of a medicament for the treatment and/or prophylaxis of a bacterial infection which is associated with the modulation of any one or more of the genes described in Table 3 of the Examples.

The present disclosure also relates to use of PBT2, or a pharmaceutically acceptable salt thereof, for the treatment and/or prophylaxis of a bacterial infection which is associated with the modulation of any one or more of the genes described in Table 3 of the Examples.

The present disclosure also relates to PBT2, or a pharmaceutically acceptable salt thereof, for use in the treatment and/or prophylaxis of a bacterial infection which is associated with the modulation of any one or more of the genes described in Table 3 of the Examples.

The present disclosure also relates to a pharmaceutical composition comprising PBT2, or a pharmaceutically acceptable salt thereof, for use in the treatment and/or prophylaxis of a bacterial infection which is associated with the modulation of any one or more of the genes described in Table 3 of the Examples.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXPERIMENTAL

### Example 1: Potency of PBT2 Alone Against Resistant Neisseria gonorrhoeae Strains

13 multidrug resistant WHO strains of Neisseria gonorrhoeae were selected to cover antimicrobial resistance phenotypes to all the antibiotics currently used. MS11 is a laboratory strain and ATCC49226 is a CLSI standard MIC control strain (CLSI. 2022. Performance Standards for Antimicrobial Susceptibility Testing, 32nd Edition. Clinical and Laboratory Standards Institute which is hereby incorporated by reference in its entirety). The MICs (minimum inhibitory concentration) of PBT2 alone in the absence of zinc or a further antimicrobial agent against these Neisseria gonorrhoea strains are summarised in Table 1.

TABLE 1
Sensitivity of Neisseria gonorrhoeae to PBT2.

| | PBT2 (mg/L) | PBT2 (mg/L) |
|---|---|---|
| Neisseria gonorrhoeae strains | Broth | Agar |
| WHO C | 0.3125 | 0.625 |
| WHO E | 0.1563 | 0.3125 |
| WHO F | 0.1563 | 0.3125 |
| WHO G | 0.1563 | 0.3125 |
| WHO K | 0.3125 | 0.625 |
| WHO L | 0.1563 | 0.625 |
| WHO M | 0.3125 | 0.625 |
| WHO N | 0.1563 | 0.625 |
| WHO O | 0.1563 | 0.625 |
| WHO P | 0.3125 | 0.625 |
| WHO X | 0.1563 | 0.625 |
| WHO Y | 0.1563 | 0.625 |
| WHO Z | 0.3125 | 0.625 |
| MS11 | 0.1563 | 0.625 |
| ATCC49226 | 0.3125 | 0.625 |

As can be seen from the results in Table 1, all Neisseria gonorrhoeae strains tested were highly susceptible to PBT2 alone (MIC 0.156-0.3125 mg/L (broth); 0.3125-0.625 mg/L (agar)).

In comparison, Table 2 shows the MICs of comparator antimicrobials, tetracycline and ciprofloxacin, against CLSI ATCC49226 Neisseria gonorrhoeae reference strain.

TABLE 2
MICs of tetracycline and ciprofloxacin for CLSI ATCC49226
Neisseria gonorrhoeae reference strain.

| | Broth | Agar |
|---|---|---|
| Neisseria gonorrhoeae ATCC49226 | Dilution | Dilution |
| Tetracycline (mg/L) | 0.25 | 0.25 |
| CLSI agar dilution | | |
| MIC QC range | | |
| 0.25-1 | | |
| Ciprofloxacin (mg/L) | 0.025 | 0.025 |
| CLSI agar dilution | | |
| MIC QC range | | |
| 0.001-0.008 | | |

### Example 2: Time-Kill Assays with Neisseria gonorrhoeae WHO Z

The Neisseria gonorrhoeae strain WHO Z (Lahra M M, Ryder N, Whiley D M.

A new multidrug-resistant strain of Neisseria gonorrhoeae in Australia. N Engl J Med 2014; 371: 1850-1) was identified in Australia in 2013. WHO Z is resistant to penicillin G, cefixime, ceftriaxone, azithromycin, ciprofloxacin and tetracycline and it carries most known resistance genes.

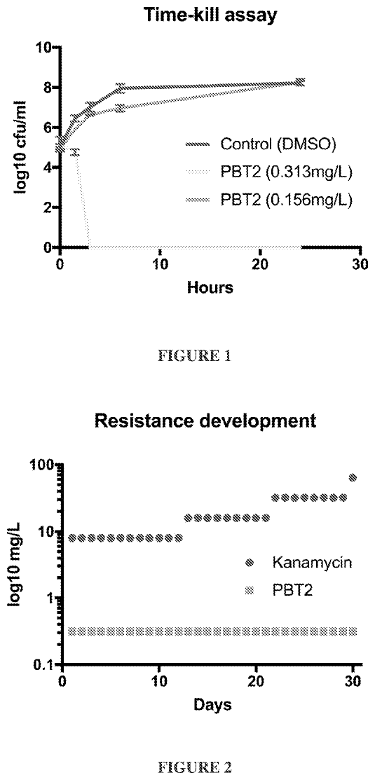

Time-kill assays were conducted (CLSI Methods of Determining Bactericidal Activity of Antimicrobial Agents; Approved Guideline, M26, V. 19, No. 18, September 1999) with PBT2 alone at MIC concentration (0.313 mg/L) and at sub-MIC concentration (0.156 mg/L). The results showed that the antibacterial activity of PBT2 at a concentration of 0.313 mg/L against MDR Neisseria gonorrhoeae strain WHO Z occurred within 3 h (FIG. 1; top line (blue) control, middle line (orange) PBT2 at 0.156 mg/L concentration, bottom line (yellow) PBT2 at 0.313 mg/L concentration).

Data from phase II clinical trials of PBT2 indicate that PBT2 is a safe-for-human-use ionophore.

The plasma drug level (AUC) of PBT2 has been reported to be 1,660 ng*h/ml (https://clinicaltrials.gov/ct2/show/results/NCT02249728) after the use of 250 mg/day for up to 72 hours, which is 5-fold higher than the PBT2 MIC (313 ng/ml) for MDR Neisseria gonorrhoeae.

### Example 3: Antibiotic Resistance Development Tests of PBT2

Resistance development was tested over a 30 day period with PBT2 and using Kanamycin as a control. No antibiotic resistance was observed after 30 consecutive daily cycles of subculture and sub-MIC PBT2 treatment (0.156 mg/L; FIG. 2; circles (blue) Kanamycin, squares (orange) PBT2).

### Example 4: Volcano Plot of Differentially Expressed Genes Between Bacteria Treated with/without PBT2

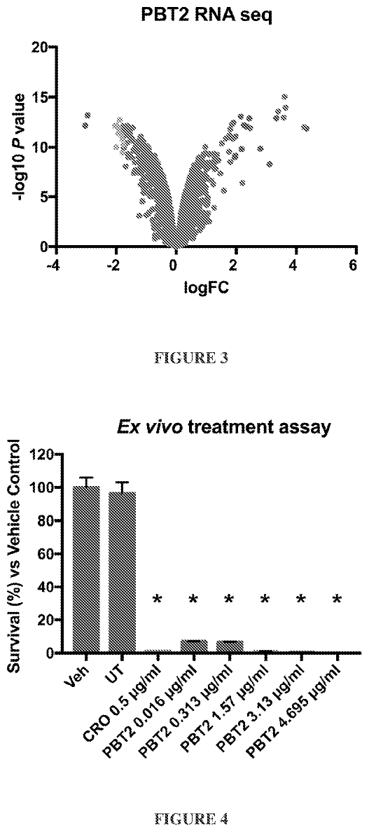

Expression profile analysis using RNA-seq was performed to investigate the mode of action of PBT2 in killing Neisseria gonorrhoeae. It was observed that sub-MIC concentrations of PBT2 (0.156 mg/L) induced extensive gene expression profile changes in MDR Neisseria gonorrhoeae. Using a cut-off of greater than, or equal to, 2-fold differential expression, and P<0.05, it was found that PBT2 induced the down-regulation of 96 genes and the up-regulation of 62 genes (FIG. 3).

The top 20 up- and down-regulated genes are shown in Table 3 and include genes involved in iron regulation and metal homeostasis. Without wishing to be bound by theory, the inventors hypothesise that this may due to the fact PBT2 is a derivative of the iron chelator clioquinol and PBT2 has the capacity to chelate a range of heavy metals including iron. Genes encoding metal homeostasis proteins (blue dots) are presented in the volcano plot (FIG. 3). Metal homeostasis was previously identified as a crucial feature of gonococcal biology, and therefore it is postulated this may be an “Achilles' heel” with respect to Neisseria gonorrhoeae ionophore sensitivity. It was also observed that a large number of genes encoding NADH-associated proteins (FIG. 3, orange dots) were down-regulated by PBT2, which may be a consequence of perturbed metal homeostasis. Seven genes were up-regulated >10 fold, including mpeR (20-fold). MpeR is an iron-repressed, AraC-like protein that regulates the mtr efflux locus (involved in antimicrobial susceptibility) in Neisseria gonorrhoeae. Up-regulation of mpeR (e. g. by PBT2) down-regulates MtrF and, in turn, would increase Neisseria gonorrhoeae sensitivity to the antimicrobial detergent, Triton X-100 (22). Thereby, it was determined the MIC of Triton X-100 in the presence of PBT2 and confirmed that PBT2 increased the sensitivity of Neisseria gonorrhoeae strain WHO Z to Triton X-100 (i.e., a decrease in the MIC from >1 g/L to 125 mg/L in the presence of0.156 mg/L (sub-MIC) PBT2). Thus, without wishing to be bound to theory, the inventors hypothesise that PBT2 may influence bacterial iron homeostasis and trigger general defence mechanisms, such as Mtr-mediated antimicrobial efflux, but the Mtr-system may be unable to protect against PBT2.

TABLE 3
Top 20 up- and down-regulated genes in MDR Neisseria gonorrhoeae
after treatment with PBT2.
Up-regulated by PBT2

| | | Fold | | |
|---|---|---|---|---|
| Gene Name | LogFC | Change | P-value | Annotation |
| *WHOZ_00302 | 4.33 | 20.16 | 1.44E−12 | AraC family transcriptional |
| | | | | regulator (MpeR) |
| WHOZ_00403 | 4.26 | 19.14 | 9.68E−13 | putative lipoprotein |
| WHOZ_02044 | 3.64 | 12.46 | 1.16E−14 | ExbD protein |
| fumC | 3.61 | 12.18 | 9.06E−16 | Fumarase C |
| WHOZ_02042 | 3.58 | 11.95 | 1.13E−13 | TonB |
| WHOZ_02043 | 3.40 | 10.53 | 2.72E−14 | ExbB oriteub |
| *WHOZ_02005 | 3.33 | 10.06 | 1.30E−13 | transferrin-binding protein-like |
| | | | | solute binding protein |
| WP_003690710.1 | 3.11 | 8.64 | 5.32E−09 | hypothetical protein |
| *WHOZ_00413 | 2.81 | 7.01 | 1.38E−10 | Thioredoxin |
| WHOZ_01985 | 2.46 | 5.49 | 1.28E−12 | hypothetical periplasmic protein |
| *WHOZ_00323 | 2.44 | 5.44 | 1.27E−13 | ABC transporter periplasmic |
| | | | | binding protein, iron related |
| *WHOZ_01152 | 2.36 | 5.13 | 8.87E−13 | hemoglobin receptor |
| WHOZ_01151 | 2.25 | 4.75 | 6.36E−13 | TonB-dependent receptor plug |
| | | | | domain-containing protein |
| WP_003690577.1 | 2.20 | 4.61 | 4.27E−07 | hypothetical protein |
| *WHOZ_01683 | 2.18 | 4.53 | 1.57E−10 | fetA - Ferric enterobactin receptor |
| *WHOZ_00412 | 2.15 | 4.43 | 8.79E−14 | putative ferredoxin |
| WHOZ_01837 | 1.97 | 3.91 | 8.32E−12 | outer membrane protein |
| WHOZ_00880 | 1.97 | 3.91 | 6.64E−10 | integral membrane protein |
| *WHOZ_01153 | 1.96 | 3.89 | 1.05E−09 | hemoglobin receptor |
| *WHOZ_00324 | 1.90 | 3.74 | 3.74E−13 | iron(III) ABC transporter permease |

Down-regulated by PBT2

| | | Fold | | |
|---|---|---|---|---|
| Gene Name | LogFC | Change | PValue | Annotation |
| *bfr | 3.03 | 8.20 | 9.79E−11 | bacterioferritin |
| *bfr | 2.95 | 7.73 | 3.47E−11 | bacterioferritin |
| *WHOZ_01546 | 2.04 | 4.11 | 9.79E−11 | NADH dehydrogenase subunit I |
| *WHOZ_01548 | 2.00 | 4.00 | 3.19E−09 | NADH dehydrogenase subunit K |
| *WHOZ_01549 | 1.98 | 3.95 | 9.79E−11 | NADH:ubiquinone dehydrogenase, |
| | | | | L subunit |
| *WHOZ_01547 | 1.93 | 3.80 | 2.40E−10 | NADH dehydrogenase subunit J |
| *WHOZ_01541 | 1.92 | 3.77 | 1.16E−10 | NADH dehydrogenase subunit E |
| *WHOZ_01550 | 1.90 | 3.73 | 1.29E−10 | Predicted HKD family nuclease |
| *WHOZ_01551 | 1.88 | 3.68 | 4.26E−11 | NADH dehydrogenase subunit M |
| *WHOZ_01545 | 1.84 | 3.58 | 3.12E−10 | NADH-quinone oxidoreductase |
| | | | | subunit H |
| *WHOZ_01542 | 1.83 | 3.57 | 1.26E−10 | NADH-quinone oxidoreductase, F |
| | | | | subunit |
| WHOZ_00293 | 1.83 | 3.56 | 1.29E−10 | Periplasmic protein |
| *nqrE | 1.80 | 3.48 | 6.72E−10 | Na(+)-translocating NADH- |
| | | | | quinone reductase subunit |
| *nqrF | 1.80 | 3.47 | 9.61E−09 | Na(+)-translocating NADH- |
| | | | | quinone reductase subunit |
| *WHOZ_01552 | 1.77 | 3.41 | 9.79E−11 | NADH dehydrogenase subunit N |
| *cysK | 1.76 | 3.38 | 2.14E−09 | Cysteine synthetase |
| WHOZ_00873 | 1.75 | 3.36 | 3.76E−09 | outer membrane protein |
| *WHOZ_01539 | 1.73 | 3.32 | 3.87E−10 | NADH dehydrogenase subunit C |
| *WHOZ_00998 | 1.72 | 3.30 | 1.29E−10 | Na(+)-translocating NADH- |
| | | | | quinone reductase subunit B |
| *WHOZ_00996 | 1.70 | 3.25 | 2.61E−10 | Na(+)-translocating NADH- |
| | | | | quinone reductase subunit D |

*Metal-associated protein.

### Example 5: Potency of PBT2 Against Neisseria gonorrhoeae Infection of Human Cervical (Pex) Cells

To assess the ability of PBT2 to cure an established gonococcal infection in a human ex vivo system, primary human cervical epithelial (Pex) cells were challenged with Neisseria gonorrhoeae strain WHO Z for 90 min and then treated with 0.1% DMSO (vehicle control), PBT2 (0.016 mg/L to 4.695 mg/L), or ceftriaxone (0.5 mg/L, positive control) for 24 h. Viable gonococci were then enumerated. In these assays, when compared to the vehicle control, less than 8% of viable gonococci were recovered from Pex cell lysates after treatment with >0.157 mg/L of PBT 2, which was of comparable effectiveness to that observed for the control antibiotic, ceftriaxone (0.5 mg/L), to which WHOZ is susceptible. PBT2-induced Pex cell cytotoxicity was not observed during the assay. The results of the ex vivo treatment assay are summarised in FIG. 4.

### Example 6: Sensitivity of Neisseria meningitidis strains to PBT2

During 2019-2020, 11 Neisseria meningitidis isolates from U.S. patients were reported by the CDC that were both beta-lactamase-producing and had ciprofloxacin resistance-associated mutation. The MIC of PBT2 against strains representing 7 serogroups of Neisseria meningitidis was determined (Peak I R, Jennings C D, Jen F E, Jennings M P. Role of Neisseria meningitidis PorA and PorB expression in antimicrobial susceptibility. Antimicrob Agents Chemother. 2014; 58(1):614-6). Similar to Neisseria gonorrhoeae, all Neisseria meningitidis strains tested were sensitive to PBT2 (MIC 0.156-0.625 mg/L (broth), 0.039-0.156 mg/L (agar)) (Table 4).

TABLE 4
Neisseria meningitides strains tested against PBT2.

| Neisseria meningitidis strains | PBT2 (mg/L) | PBT2 (mg/L) |
|---|---|---|
| (serogroup) | Broth | Agar |
| Z2491 (A) | 0.1563 | 0.039 |
| MC58 (B) | 0.1563 | 0.039 |
| 8013 (C) | 0.1563 | 0.078 |
| PMC1 (X) | 0.1563 | 0.039 |
| PMC2 (Z/29E) | 0.1563 | 0.039 |
| PMC10 (Y) | 0.625 | 0.156 |
| PMC19 (W) | 0.1563 | 0.078 |

### Example 7: Sensitivity of Non-Typeable Haemophilus influenzae (NTHi) Strains to PBT2 Alone

The MIC of PBT2 against five non-typeable Haemophilus influenzae (NTHi) strains was determined (CLSI. 2022. Performance Standards for Antimicrobial Susceptibility Testing, 32nd Edition. Clinical and Laboratory Standards Institute). All Haemophilus influenzae tested were found to be sensitive to PBT2. The results are summarised in Table 5.

TABLE 5
Non-typeable Haemophilus influenza
(NTHi) strains tested against PBT2.

| | PBT2 (mg/L) |
|---|---|
| NTHi strains | Broth |
| 2019* | 2.5 |
| 723* | 1.25 |
| 86-028NP* | 2.5 |
| R2866* | 2.5 |
| ATCC49766* | 0.3125 |

*2019 is a clinical isolate obtained from a patient with chronic obstructive pulmonary disease, 723 was originally isolated from a child with otitis media, 86-028NP is a pathogenic nontypeable strain isolated from a patient with chronic otitis media, both 86-028NP and R2866 are antimicrobial (ampicillin) resistant strains (Smith, H. K. et al. (2013) Affect of anaerobiosis on the antibiotic susceptibility of H. influenzae, BMC Research Notes 6: 241).

### Example 8: Sensitivity of Campylobacter jejuni Strains to PBT2 Alone

The MIC of PBT2 against three Campylobacter jejuni strains was determined. All strains tested were sensitive to PBT2. The results are summarised in Table 6.

TABLE 6
Campylobacter jejuni strains tested against PBT2.

| | PBT2 (mg/L) |
|---|---|
| Campylobacter jejuni strains | Broth |
| 81-176* | 0.156 |
| NCTC 11168* | 0.313 |
| ATCC 33560 | 0.156 |

*81-176 was originally isolated from a human disease outbreak in 1985 and has been shown to cause inflammatory colitis in two human challenge studies (Black, R. E. et al. (1988). Experimental Campylobacter jejuni infection in humans. J. Infect. Dis. 157: 472-479); NCTC 11168 the original clonal clinical isolate from chicken (Gaynor, E. c. et al. (2004). The Genome-Sequenced Variant of Campylobacter jejuni NCTC 11168 and the Original Clonal Clinical Isolate Differ Markedly in Colonization, Gene Expression, and Virulence-Associated Phenotypes. J Bacteriol. 186(2): 503-517)

### Example 9: Sensitivity of Helicobacter pylori Strains to PBT2 Alone

The MIC of PBT2 against four Helicobacter pylori 26695 strains was determined. The strains tested were sensitive to PBT2. The results are shown in Table 7.

TABLE 7
MIC data for Helicobacter pylori strains tested against PBT2.

| | PBT2 (mg/L) |
|---|---|
| H. Pylori strains | Agar |
| 26695 | 1.25 |
| ATCC 43504 | 2.5 |
| CH426^{#} | 1.25 |
| CH428^{#} | 2.5 |

^{#}CH426 and CH428 were clinically isolated by gastric biopsy (Nguyen, A. N. T. et al. (2022) Recombination resolves the cost of horizontal gene transfer in experimental populations of Helicobacter pylori. Proc Natl Acad Sci U S A 119, e2119010119. 10.1073/pnas.2119010119). Both CH426 and CH428 are MDR strains which are resistant to amoxicillin, metronidazole and clarithromycin.

### Example 10: Brief Synthetic Overview for PBT2

As discussed herein, PBT2 has been known for some time and a straightforward synthetic route is known and available to the person of skill in the art at least in Bohlmann L, et al. 2018. Chemical synergy between ionophore PBT2 and zinc reverses antibiotic resistance. mBio 9:e02391-18. https://doi.org/10.1128/mBio.02391-18, which is hereby incorporated by reference in its entirety. Nonetheless, a brief overview of the synthesis is provided below from that article.

PBT2 (compound 3, below) may be synthesized following the reaction conditions shown below (previously described in: Barnham K J, July 2008. Preparation of 8-hydroxyquinolines for treatment of neurological conditions; US patent publication no. 20080161353A1). Initially, oxidation of the methyl side chain of 5,7-dichloro-2-methylquinolin-8-ol (compound 1) is achieved by heating compound 1 with selenium dioxide in 1,4-dioxane to provide the required aldehyde (compound 2), in quantitative yield. The resultant crude product is then further reacted with dimethylamine-hydrochloride in 1,2-dichloroethane and triethylamine to yield a product that is reduced in situ, by treatment with sodium triacetoxyborohydride, to provide the free amine of PBT2 as an oil. Upon acidification of the free amine with HCl, PBT2 hydrochloride-salt (compound 3) may be obtained in 81% yield.

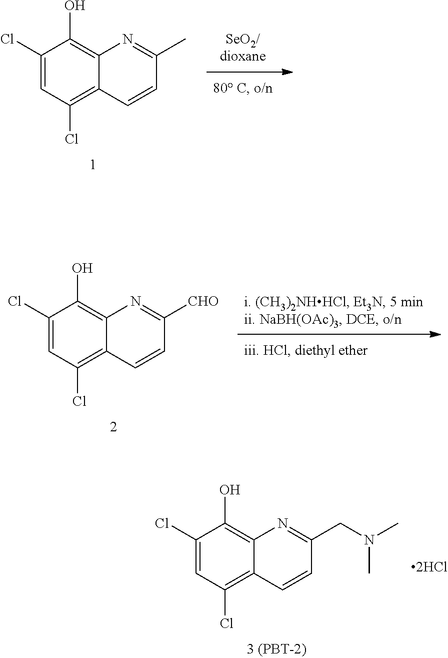 [SMILES: Cc1ccc2c(Cl)cc(Cl)c(O)c2n1 ; O=Cc1ccc2c(Cl)cc(Cl)c(O)c2n1 ; CN(C)Cc1ccc2c(Cl)cc(Cl)c(O)c2n1]

1. A method for the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections, in a subject, comprising administering an effective amount of PBT2, or a pharmaceutically acceptable salt thereof, to the subject.

2. Use of PBT2, or a pharmaceutically acceptable salt thereof, in the preparation of a medicament for the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections.

3. Use of PBT2, or a pharmaceutically acceptable salt thereof, for the treatment and/or prophylaxis of a bacterial infection selected from the group consisting of: Neisseria gonorrhoeae, Neisseria meningitidis, nontypeable Haemophilus influenzae, Campylobacter and Helicobacter pylori infections.

4. A method for the treatment and/or prophylaxis of a bacterial infection selected from the group consisting of: Neisseria meningitidis, nontypeable Haemophilus influenzae, Campylobacter and Helicobacter pylori infections, in a subject, comprising administering an effective amount of PBT2, or a pharmaceutically acceptable salt thereof, to the subject.

5. Use of PBT2, or a pharmaceutically acceptable salt thereof, in the preparation of a medicament for the treatment and/or prophylaxis of a bacterial infection selected from the group consisting of: Neisseria meningitidis, nontypeable Haemophilus influenzae, Campylobacter and Helicobacter pylori infections.

6. Use of PBT2, or a pharmaceutically acceptable salt thereof, for the treatment and/or prophylaxis of a bacterial infection selected from the group consisting of: Neisseria meningitidis, nontypeable Haemophilus influenzae, Campylobacter and Helicobacter pylori infections.

7. The method or use of any one of the preceding claims, wherein PBT2, or a pharmaceutically acceptable salt thereof, is the sole active antibacterial agent.

8. The method or use of any one of the preceding claims, wherein PBT2, or a pharmaceutically acceptable salt thereof, is administered in the absence of zinc.

9. The method or use of any one of the preceding claims, wherein the PBT2, or a pharmaceutically acceptable salt thereof, is administered by a route selected from the group consisting of oral, systemic, topical, subcutaneous, intravenous, intraparenteral, intra-synovial, intravaginal, intraocular, transbuccal, transmucosal, and transdermal administration.

10. The method or use of any one of the preceding claims, wherein the PBT2, or a pharmaceutically acceptable salt thereof, is administered in the absence of both zinc and a further antibacterial agent.

11. The method or use of any one of the preceding claims, wherein PBT2, or a pharmaceutically acceptable salt thereof, is used or administered to the subject in an amount to achieve a peak blood concentration of between about 0.005 to about 25 mg/L, between about 0.005 to about 20 mg/L, between about 0.005 to about 15 mg/L, between about 0.005 to about 10 mg/L, between about 0.005 to about 5 mg/L, between about 0.005 to about 4 mg/L, between about 0.005 to about 3 mg/L, between about 0.01 to about 25 mg/L, between about 0.01 to about 20 mg/L, between about 0.01 to about 15 mg/L, between about 0.01 to about 10 mg/L, between about 0.01 to about 5 mg/L, between about 0.01 to about 4 mg/L, between about 0.01 to about 3 mg/L, between about 0.5 to about 25 mg/L, between about 0.5 to about 20 mg/L, between about 0.5 to about 15 mg/L, between about 0.5 to about 10 mg/L, between about 0.5 to about 5 mg/L, between about 0.5 to about 4 mg/L, between about 0.5 to about 3 mg/L, between about 1.0 to about 25 mg/L, between about 1.0 to about 20 mg/L. between about 1.0 to about 15 mg/L, between about 1.0 to about 10 mg/L, between about 1.0 to about 5 mg/L, between about 1.0 to about 4 mg/L, or between about 1.0 to about 3 mg/L.

12. The method or use of any one of the preceding claims, wherein the subject is a vertebrate animal, optionally a mammal, optionally a human subject in need of treatment and/or prophylaxis for said bacterial infection.

13. The method or use of any one of the preceding claims, wherein the Campylobacter is selected from the group consisting of: Campylobacter jejuni, Campylobacter coli, Campylobacter abortis, Campylobacter hepaticas, Campylobacter upsaliensis, Campylobacter helveticus and Campylobacter fetus.

14. The method or use of any one of the preceding claims, wherein the Campylobacter is Campylobacter jejuni.

15. A pharmaceutical composition comprising an effective amount of PBT2, or a pharmaceutically acceptable salt thereof, and a pharmaceutically acceptable excipient, wherein the composition does not comprise zinc or a further antibacterial active.

16. The pharmaceutical composition according to claim 15, wherein the pharmaceutical composition is an antibacterial composition which consists of or consists essentially of PBT2, or a pharmaceutically acceptable salt thereof, as the antibacterial active.

17. The pharmaceutical composition according to claim 15 or claim 16, wherein the pharmaceutical composition consists of or consists essentially of PBT2, or a pharmaceutically acceptable salt thereof, and one or more pharmaceutically acceptable excipients, diluents or carriers.
